(19) United States
(12) Patent Application Publication
(10) Pub. No.: US 2004/0127571 A1
(43) Pub. Date: Jul. 1, 2004

(54) Method of Treating Leukemia with a Combination of Suberoylanilide Hydromaxic Acid and Imatinib Mesylate

(75) Inventors: Kapil N. Bhalla, Tampa, FL (US); Ramadevi Nimmanapalli, Tampa, FL (US)

Correspondence Address: SMITH & HOPEN PA, 15950 BAY VISTA DRIVE, SUITE 220, CLEARWATER, FL, 33760

(73) Assignee: UNIVERSITY OF SOUTH FLORIDA, Tampa, FL (US)

(21) Appl. No.: 10/605,283

(22) Filed: Sep. 19, 2003

Related U.S. Application Data
(60) Provisional application No. 60/319,563, filed on Sep. 19, 2002.

(51) Int. Cl. A61K 31/19

(52) U.S. Cl. 514/575

(57) ABSTRACT

A method for inducing apoptosis, or increasing the rate or extent of apoptosis, in target cells. The method comprises the steps of contacting the cancer cells with an apoptosis-inducing amount of a tyrosine kinase inhibitor, imatinib mesylate, and a histone deacetylase inhibitor, Suberoylanilide Hydromaxic Acid (SAHA). The method is applicable to ameliorating the resistance of the accelerated and blast phases of CML (CML-BC) to imatinib mesylate.

### CROSS-REFERENCE TO RELATED APPLICATION

This application claims priority from U.S. provisional application Serial No. 60/319,563, entitled: “Suberoylanilide Hydroxamix Acid Treatment of Leukemia Cells”, filed Sep. 19, 2002.

### BACKGROUND OF INVENTION

1. Field of Invention

This invention relates to a treatment for leukemia. More specifically, the invention is a method for increasing the extent or rate of apoptosis in a subject, particularly in association with leukemia therapy protocols.

2. Background of the Invention

Apoptosis, also called programmed-cell-death, is a normal process by which cells self-destruct. Apoptosis, when properly regulated, is a beneficial process by which cells disintegrate into compact units which are then phagocytized by neighboring cells. Apoptosis can be triggered by a loss of contact with neighboring cells, smothering of the cell surface, or DNA damage. Once triggered, the process includes the contraction and concentration of the cytoplasm, chromatin condensation and pyknosis, and segmentation of the nucleus. The DNA in the nucleus degrades into nucleosomal fragments coupled with cellular fission resulting in the formation of apoptic bodies. The cellular fragments are “eaten,” phagocytized, by neighboring cells. This process is a method of protection for cells that are malfunctioning. In particular, induction of apoptosis is beneficial if the process causes the death of abnormal cells, such as cancer cells.

Resistant forms of cancer have acquired the ability to avoid at least some triggers of apoptosis. Downregulation of apoptosis is involved in a number of diseases and conditions, particularly leukemia. By gaining control of apoptotic pathways, it is possible to diminish the occurrence of cancerous cells in a subject.

Studies have shown that 95% of all chronic myeloid leukemia (CML) and 20% of adult acute lymphoblastic leukemia cases bear the Bcr-Abl fusion oncogene. The oncogene creates the Bcr-Abl protein. The Bcl proteins are associated with membrane activity and play a vital role n signaling and controlling apoptosis. As a result the overexpression of this protein can prevent apoptosis in damaged cells in which apoptosis would otherwise be triggered. Bcr-Abl positive cells are specifically resistant to apoptosis induced by DNA damage. It is known that dys-regulated activity of the Bcr-Abl tyrosine kinase in the cytosol activates several molecular mechanisms that inhibit apoptosis, and as a result contribute to the drug-resistance of Bcr-Abl positive leukemia blasts.

The advent of imatinib mesylate brought about great step in the fight against leukemia. Imatinib mesylate is a selective inhibitor of the Bcr-Abl tyrosine kinase. Imatinib mesylate works to bind the Bcr-Abl receptors thereby blocking adenosine triphosphate (ATP) binding. The Bcr-Abl protein cannot carry on its kinase activity without the energy supplied by the ATP molecule.

Imatinib mesylate has shown impressive results in selectively inducing apoptosis in chronic phase CML, however the accelerated and blast phases of CML (CML-BC) have shown to be resistant to imatinib mesylate. Therefore, a need exists to identify additional agents that work with imatinib mesylate, or alone, to increase the rate or extent of apoptosis in target living cells.

### SUMMARY OF INVENTION

It has been discovered that the rate or extent apoptosis can be induced in living cells by contacting the target cells with an apoptosis-inducing amount of a tyrosine kinase inhibitor and a histone deacetylase inhibitor that: a. inhibits the binding of ATP with the BCR-ABL protein, thus preventing the Bcr-Abl protein from carrying out its kinase activity; b. induces hyper-acetylation of the amino terminal lysine residues of the core nucleosomal histones and of specific transcriptional regulators.

In another embodiment the invention relates to a method of ameliorating the resistance of the accelerated and blast phases of CML (CML-BC) to imatinib mesylate, comprising the steps of administering to the subject a combination tyrosine kinase inhibitors and histone deacetylase inhibitors in an amount effective to selectively accumulate in target cells and contacting the target cells with an apoptosis-inducing amount of a tyrosine kinase inhibitor and a histone deacetylase inhibitor.

Treatment with the tyrosine kinase inhibitor imatinib mesylate (GLEEVEC, formerly known as STI571 or CGP57148B) has been shown to selectively inhibit the growth of, and induce apoptosis in, leukemia cells that possess Bcr-Abl tyrosine kinase activity. Although treatment with imatinib mesylate alone has been successful with cells in the chronic phase, the accelerated and blast crisis phases of chronic myeloid leukemia (CML-BC) and Bcr-Abl positive acute lymphoblastic leukemia (ALL) prove to be highly resistant to imatinib mesylate. However, CML cells exposed to concentrations of (1.0 uM to 10.0 uM) of the histone deacetylase inhibitor Suberoylanilide Hydromaxic Acid (SAHA) and imatinib mesylate for 48 hours drastically increased apoptosis, on a dose dependant basis, in CML and imatinib mesylate-refractory (CML-BC) cells.

### BRIEF DESCRIPTION OF DRAWINGS

For a fuller understanding of the nature and objects of the invention, reference should be made to the following detailed description, taken in connection with the accompanying drawings, in which:

FIG. 1 is a diagrammatic view of the method according to the invention.

FIG. 2 shows levels of apoptosis after exposure to SAHA, in the indicated concentrations for 48 hours.

FIG. 3 shows results of co-treatment of SAHA and imatinib mesylate, in indicated concentrations, for 48 hours (FIGS. 3A 3C) and 24 hours (FIG. 3D).

### DETAILED DESCRIPTION

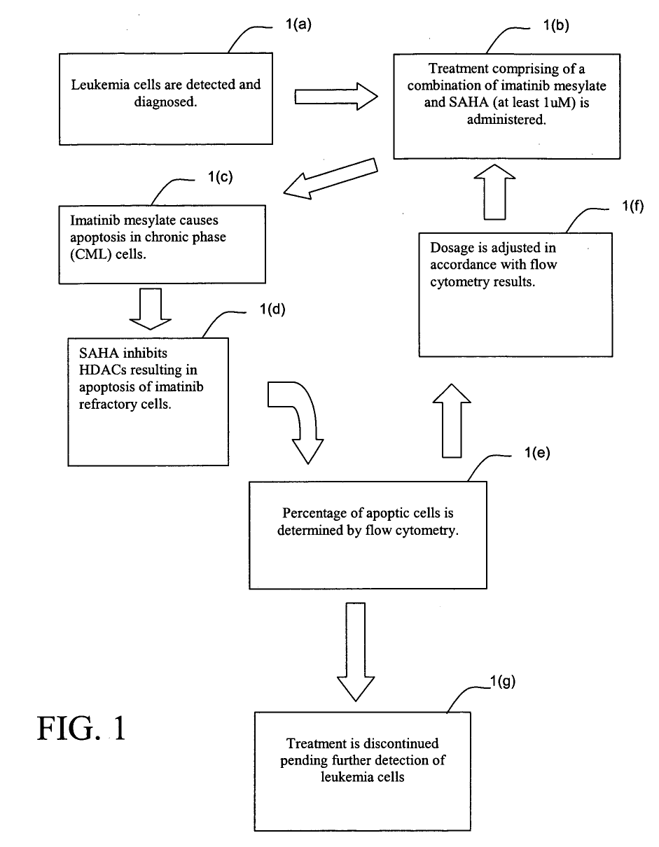

An embodiment of the invention includes detecting and identifying target leukemia cells within a patient. Turning to FIG. 1, cells are diagnosed and treatment is administered comprising of a combination of imatinib mesylate and SAHA substantially concurrently 20. Once administered imatinib mesylate causes apoptosis in chronic phase CML cells via known pathways 30. Concurrently, SAHA inhibits the activity of histone deacetylases in imatinib refractory cells, resulting in apoptosis 40. The percentage of apoptic cells can then be determined by flow cytometry 50, wherein samples of mononuclear cells are passed through a beam of laser light. On a result-dependent basis the dosage can be adjusted 60 and re-administered 20, the dosage can be adjusted on either an increased concentration or increased exposure basis. If satisfactory results are achieved treatment is discontinued pending further detection or increase in CML cell activity 70.

SAHA is a known histone deacetylase (HDAC) inhibitor. HDAC inhibitors induce hyper-acetylation of the amino terminal lysine residues of the core nucleosomal histones and of specific transcriptional regulators. Such activity has been implicated in chromatin remodeling and transcriptional upregulation of cell-cycle and differential regulatory genes, such as p21, thus resulting in apoptosis, cell death, of cancer and leukemia cells.

With regard to the treatment of leukemia, the Bcl-Abl protein is of particular interest. Bcl-2 (B cell lymphoma gene-2) proteins are associated with membrane activity and play a key role in controlling, or preventing, apoptosis. The overexpression of this protein can prevent apoptosis in damaged cells, such as leukemia cells, and increase the cells resistance to treatments such as imatinib mesylate.

Imatinib mesylate is a tyrosine kinase inhibitor. Imatinib mesylate works to induce apoptosis in cancerous cells by binding to the malfunctioning Bcr-Abl receptors and blocking ATP. Thus Bcr-Abl cannot continue to perform thus inducing apoptosis. Although imatinib mesylate has shown impressive results in selectively inducing apoptosis in chronic phase CML, the accelerated and blast phases of CML (CML-BC) have shown to be resistant to imatinib mesylate.

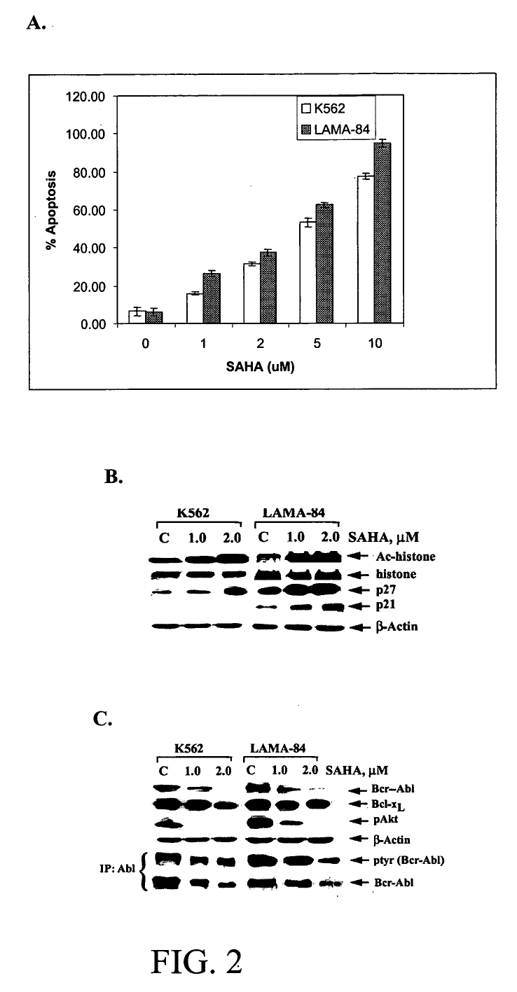

Target cell exposure to 1.0 to 10.0 uM SAHA for 48 hours induces a dose-dependent increase in the apoptosis of K562 and Lama-84 cells (FIG. 2A). Treatment with 1.0 to 5.0 uM SAHA for 48 hours increased the percentage of K562 in the G1 (the gap in the cell division cycle between mitosis and synthesis) phase of the cell cycle in a dose-dependent manner.

Moreover, exposure to SAHA increases the acetylation of histones (H3) and the expression of p21 (FIG. 2B). Treatment with SAHA induced p27 expression concurrently in both K562 and LAMA cells (FIG. 2B). Previously, SAHA had been shown not to induce acetylation of histones of the chromatin associated with p27 (gene) or increase its transcription. SAHA downregulates the levels and auto-phosphorylation, the addition of a phosphate group, of Bcr-Abl (FIG. 2C).

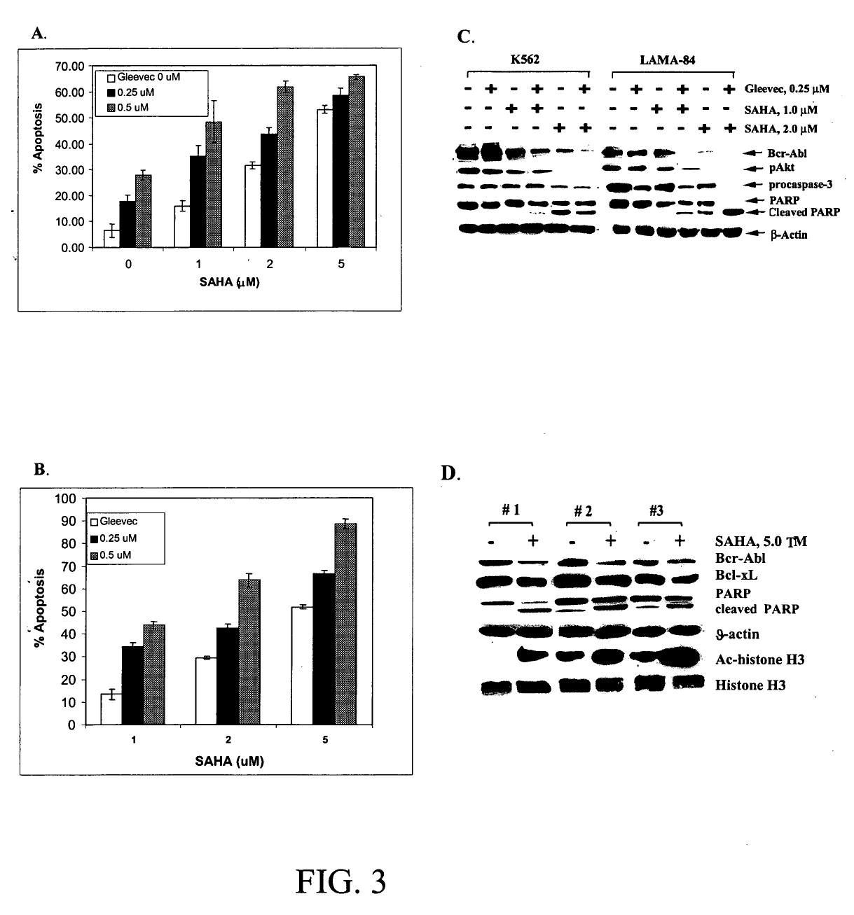

Combined treatment with SAHA and imatinib mesylate for 48 hours induced more apoptosis of target cells as compared to the treatment with either agent alone as shown in FIGS. 3A and 3B. This increase is apoptotic effect is associated with an increased decline in the levels of Bcr-Abl, p-Akt and procaspase 3 (FIG. 2C). Effectiveness of the combination of the agents was effective on an exposure-dependent, shorter exposures to the combination induced a lesser apoptotic effect.

Therefore, treatment of leukemia by combining exposure of the target cells to SAHA and imatinib mesylate, concurrently, clearly shows that the downregulation of Bcr-Abl, caused by SAHA, induces apoptosis in imatinib refractory (resistant) cells. Collectively, combined treatment with SAHA and imatinib mesylate against the advanced stages of CML, in addition to the effects of SAHA on imatinib refractory CML-BC, is a superior method for the treatment of leukemia.

The pharmaceutical composition of the invention may also comprise a pharmaceutically acceptable carrier, methods of formulation are well-known in the art. Accordingly, the invention includes a chemical composition for inducing apoptosis in cancer cells including a tyrosine kinase inhibitor, imatinib mesylate, and a histone deacetylase inhibitor, SAHA. This invention provides a method of treating living cells, which include, but is not limited to, humans and other mammals. Factors such as the method of administration, the patient's age, severity of the disease, and similar variables are to be considered when deciding on a subject-specific dosage.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

1. A method for inducing apoptosisin cancer cells, the method comprising the steps of contacting the living cells with a tyrosine kinase inhibitor and a histone deacetylase inhibitor.

2. The method of claim 1 wherein the tyrosine kinase inhibitor is imatinib mesylate.

3. The method of claim 1 wherein the histone deacetylase inhibitor is suberoylanilide hydromaxic acid.

4. The method of claim 1 wherein the tyrosine kinase inhibitor is imatinib mesylate and the histone deacetylase inhibitor is suberoylanilide hydromaxic acid.

5. Method of claim 1 wherein the living cells are exposed to the tyrosine kinase inhibitor and the histone deacetylase inhibitor for about 48 hours.

6. Method of claim 1 wherein the cancer cells are leukemia cells.

7. Method of claim 1 wherein the cancer cells are imatinib mesylate refractory.

8. A method of potentiating a cytotoxic effect of a tyrosine kinase inhibitor-based treatment comprising the steps of contacting target cells with a histone deacetylase.

9. The method of claim 8 wherein the tyrosine kinase inhibitor-based treatment comprises imatinib mesylate.

10. The method of claim 8 wherein the histone deacetylase inhibitor is suberoylanilide hydromaxic acid.

11. Method of claim 8 wherein the target cells are leukemia cells.

12. Method of claim 8 wherein the target cells are imatinib mesylate refractory.

13. A chemical composition for inducing apoptosis in cancer cells comprising a tyrosine kinase inhibitor and a histone deacetylase inhibitor.

14. The chemical composition of claim 13 wherein the tyrosine kinase inhibitor is imatinib mesylate.

15. The chemical composition of claim 13 wherein the histone deacetylase inhibitor is suberoylanilide hydromaxic acid.

16. The chemical composition of claim 13 wherein the tyrosine kinase inhibitor is imatinib mesylate and the histone deacetylase inhibitor is suberoylanilide hydromaxic acid.
